(19) United States
(12) Patent Application Publication
(10) Pub. No.: US 2015/0075528 A1
(43) Pub. Date: Mar. 19, 2015

(54) METHOD AND APPARATUS FOR TANGLE-FREE OXYGEN LINES

(71) Applicant: Susan Leeds Kudo, Pacific Palisades, CA (US)

(72) Inventor: Susan Leeds Kudo, Pacific Palisades, CA (US)

(21) Appl. No.: 14/031,006

(22) Filed: Sep. 18, 2013

(51) Int. Cl. A61M 16/08; A62B 9/04; A61M 16/10

(52) U.S. Cl. CPC A61M 16/0875; A61M 16/1005; A62B 9/04
USPC 128/204.18

(57) ABSTRACT

The tangle-free, kink-free oxygen supply extensible tubing uses a helical accordion support structure on which the oxygen supply tubing is attached. The helical accordion backbone has some tendency to spring back, but this is not relied upon to extend and return the oxygen supply tubing it carries. The carrier structure keeps the oxygen tubing from getting tangle or kinked. A centrally located retractable line, which is located within the center of the helical accordion structure, acts as the retracting mechanism, as it tends to allow the accordion structure to be pulled into an extended position, while upon rewinding the central cord, the helical accordion support structure can be pushed toward a collapsed position without the oxygen supply line tubing becoming tanked, kinked, and thereby blocked. As a safety feature , so the oxygen line does not encounter and strain between it and the patient, a chest-harness has attachment to the helical accordion support structure.

This invention is an improvement on U.S. Pat. No. 8,460,272, issued on Jan. 11, 2013. U.S. Pat. No. 8,460,272 is incorporated by reference.

### FIELD OF THE INVENTION

The invention relates to medical oxygen lines which won't kink or tangle and which can extend and retract over a distance of many feet.

### BACKGROUND OF THE INVENTION

Medical patients often require oxygen lines, which may be attached to a stationary source as in a hospital or which may be attached to a portable source such as a small oxygen bottle.

One goal which doesn't appear to be satisfactorily solved is to make the oxygen line tangle and kink-free when it is long enough to allow a patient some reasonable freedom of movement.

An ideal approach to solving this problem would to have an extensible oxygen tube that could extend and return to its place of origin. This ideal approach would use existing oxygen tubing.

One difficulty can be noted up front: the oxygen tubing is very flexible and relatively of small diameter and relatively thin walled. This difficulty may preclude some direct approaches to answering the question of how to translate the desired result into a workable apparatus.

It is of interest to review prior art. In this conjunction it is noted that at least in one's typical experience in a modern hospital, no such extensible kink-free and tangle-free oxygen lines have been encountered. The oxygen lines are relatively short. This is based on the inventor's anecdotal experiences.

### SUMMARY OF THE INVENTION

This mechanical composition invention fulfills a long felt need. Although a number of inventors have turned their hands to proving a solution to the problem outlined in the “Background of the Invention” section above, and no doubt many Medical Doctors and scientists, engineers and inventors have been subject to hospitalization or observations of those hospitalized, the invention herein, if it were obvious, would have already been invented. Therefore, hindsight alone would not be enough to render this invention obvious.

The invention herein uses a helical accordion support structure (“backbone”) backbone on which the oxygen. tubing is attached or held. The helical accordion backbone has some tendency to spring back, but this is not relied upon to extend and return the oxygen. tubing it carries. The carrier structures keeps the oxygen tubing from getting tangle or kinked.

Instead a centrally located line retractable line, which is located within the center of the helical accordion structure, acts as the retracting mechanism, as it tends to allow the accordion structure to be pulled into an extended position, while upon rewinding the central cord, the helical accordion support structure.

An additional feature to help insure safety, so the oxygen line does not pull lose from the patient is a light-weight chest harness. The light weight chest harness typically uses a Velcro closure.

### BRIEF DESCRIPTION OF THE DRAWINGS

For a more complete understanding of the present invention, and the advantages thereof, reference is now made to the following descriptions taken in conjunction with the accompanying drawings, in which:

FIG. 1 (prior art) shows a helical accordion carrying structure;

FIG. 2 shows a helical accordion carrying structure with an oxygen line in place;

FIG. 3 shows the central retractable line located through the center of the helical accordion support structure, with attachments at oxygen supply and control element;

FIG. 4 shows a light-weight chest harness in place and no part of the living person indicated by dotted lines is part of the invention.

### DETAILED DESCRIPTION OF THE BEST MODE OF THE INVENTION

The following description is of the best mode presently contemplated for carrying out the invention. This description is not to be taken in a limiting sense, but is merely made for the purpose of describing the general principles of the invention.

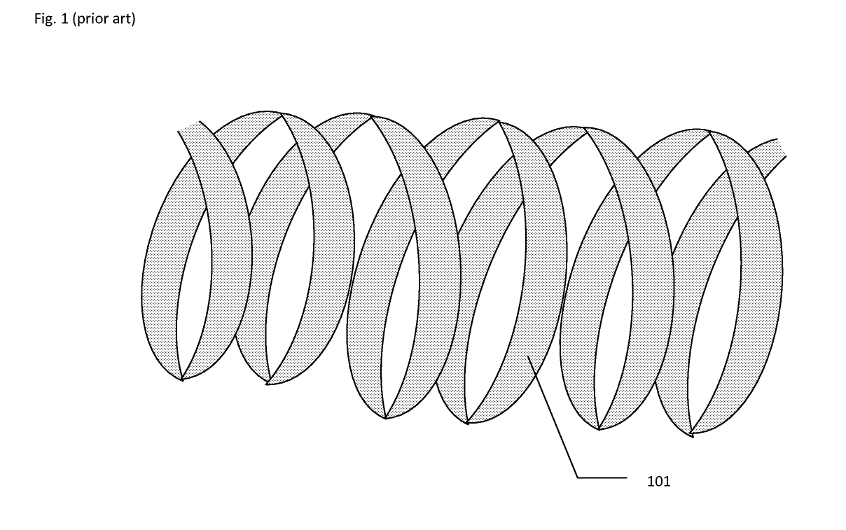

FIG. 1 shows a helical accordion carrying structure. The purpose of the helical accordion is to provide a support for an oxygen line of the type used for medical purposes. One aspect to maintaining a usefully operational oxygen supply line is to keep it from kinking-up and acquiring a kink which blocks oxygen from flowing through the kink.

FIG. 1 (prior art) shows the helical accordion support structure 101. It resembles a toy sold under a trademark name of “Slinky”, U.S. Pat. No. 2,415,012, 1947 issued to R. T. James. Prototypes of this invention were made with a commercially bought metal “Slinky.” Two different diameter plastic versions were also used. The purpose of the helical accordaon support structure is to keep the i. v. drip line from tangling.

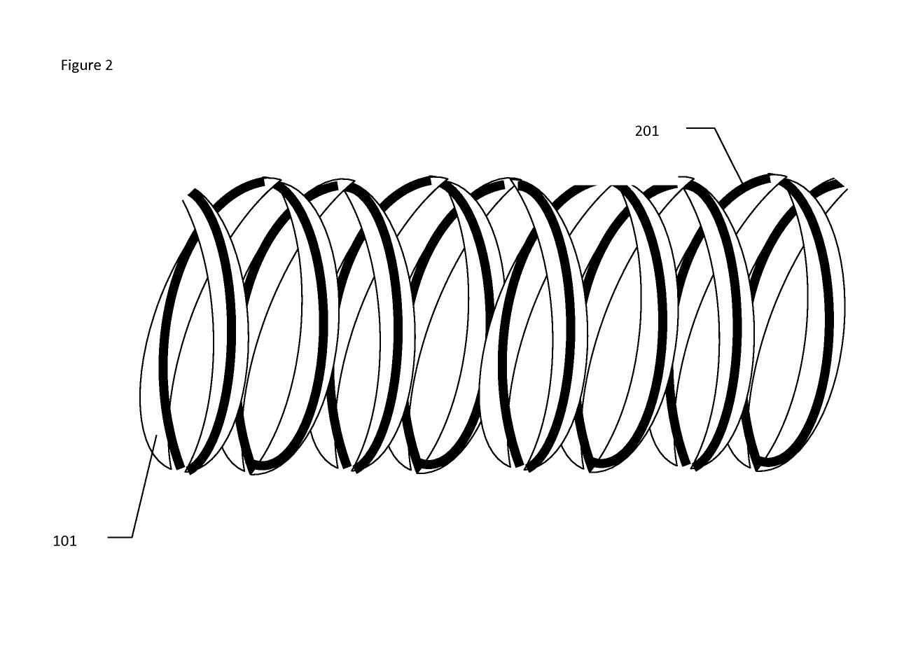

FIG. 2 shows the oxygen p line 201 in relationship to the helical support structure. It is very important that the oxygen line does not become twisted and kinked up. Otherwise the oxygen line would not be able to suppoxygen to the patient.

The helical accordion support structure 101 provides an indexing topological base such that the sequential ordering of the oxygen line 201 is maintained: a first point remains ordered behind a second point in a linear ordering of the oxygen supply line.

The accordion support structure is necessary but not sufficient to have the extensible oxygen line apparatus operate.

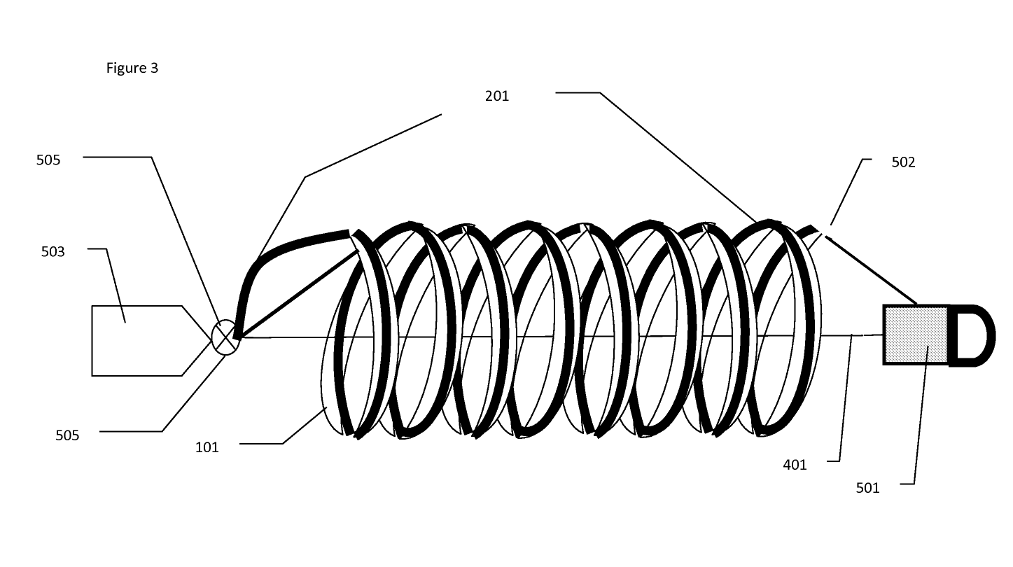

FIG. 3 shows a “central line” 401 through the center of the helical accordion support structure 101. The term “central line” 401 is used as specifically shown in these figures and is not another oxygen supply line. This central line 401 serves as a support for the helical accordion support structure 101. It also serves to allow the helical accordion support structure 101 to be pulled open and to be pulled shut. The springiness of the accordion aspect of the helical accordion support structure 101 is fairly weak. Also the helical accordion support structure 101 tends to sag even without supporting any oxygen supply line 201. The central control line 401 tends to counteract this sag.

The springiness of the accordion aspect of the helical accordion support structure 101 is easily pulled apart and easily aided in compression by attaching the end 502 of the helical accordion support structure 101, which may be closest to the patients nose and mouth, to an control element 501 (See Fig.).

As shown in FIG. 3, the other end of the central line 401 is anchored to an anchor point 505 of an oxygen supply point 503, which may be an oxygen bottle or a hospital bed oxygen supply node. The central line 401 and the control element 501 are comparable to a commercially available variable length “dog leash,” known in the arts, such as one trademarked “flexi”, model classic 1. These commercially available adjustable dog leashes essentially can rewind the dog leash line, but don't “haul the dog in,” because the rewind spring of this control element 501 is not super-strong. The control element may be based on the adjustable dog leash described here, which also has a thumb trigger for allowing the line, corresponding to the central line 401 to rewind. Any similar mechanism is suitable for the control element 501, even if it is repackaged.

By allowing the control element 501 to pay out line length, a person can walk forward and the accordion aspect of the helical accordion support structure 101 will open up, carrying with it the oxygen supply line. 201. Thus the oxygen supply line 201 extends out carried by the accordion structure of helical accordion support structure 101. As the person walks back toward the oxygen supply point 503 and allows control element 501 to allow the central line to retract, the accordion aspect of the helical accordion support structure 101 tend to close up, contracting with the oxygen supply line 201 attached to it.

The refraction ability of central line 401 rewinding into the control element 501 is fairly weak, but combined with the slight compression ability of the accordion aspect of the helical accordion support structure 101, the helical accordion support structure 101 with the attached oxygen supply line 201 are easily pushed backed to a compressed situation where the total length of the helical accordion support structure 101 is shortened to an fully compressed condition. Similarly, the helical accordion support structure 101 and the attached oxygen supply line are easily extensible by releasing the central line 401 by causing the control element to release and pulling on the control element which is attached to one end of the helical accordion support structure 101.

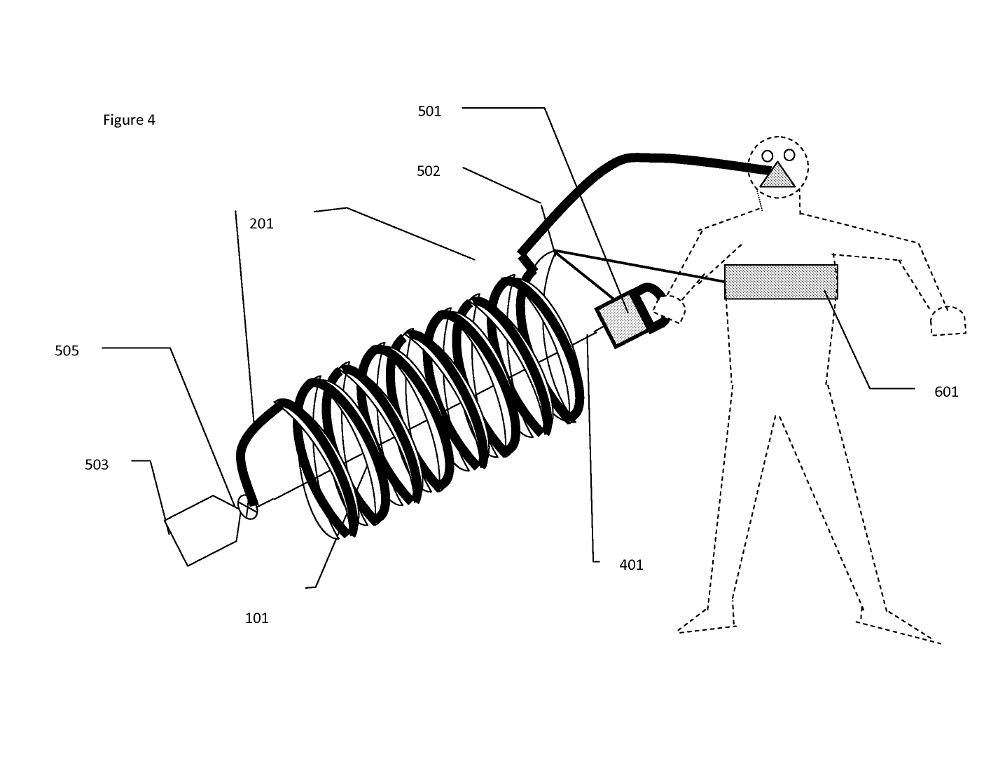

FIG. 4 shows a chest harness 601. The dotted outline of the human patient is not part of the invention. The central line 401 is shown with the helical accordion structure 101, the oxygen line 201. The oxygen supply point 503 shown along with an attachment point 505. An second attachment point 502 for the helical accordion structure 101 is shown together with the control element 501.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention.

It is claimed that:

1. An apparatus for an tangle-free, kink-free extensible oxygen supply line, comprising:
an helical accordion support structure;
an oxygen supply line attachable to the helical accordion support structure;
an attachment means wherein the oxygen supply line is attached to the helical accordion support structure;
a controllable central line; and
a controller for selectively releasing the controllable central line to be released or to be rewound.

2. The apparatus of claim 1, further comprising a chest-harness safety feature, wherein a chest-harness attachable to the chest of a patient is attached to the helical accordion support structure and wherein said configuration will prevent severe stress on the oxygen supply line in the event the central control line element is dropped.

3. The apparatus of claim 1, further comprising a bracket for a kit which is adapted to attached to an oxygen supply node wherein a screw tightened bracket means provides an anchor for the central line.

4. A method for an tangle-free, kink-free extensible oxygen supply line, comprising:
supporting an oxygen supply line by attaching the oxygen supply line to a helical accordion-like support structure;
expanding the extensible length of the oxygen supply line by expanding the helical accordion support structure; by pulling on it;
contracting the extensible length of the oxygen supply line by contracting the helical accordion support structure; by pulling on it;
preventing the sagging of the helical supporting structure by having a first end of central line attached to an oxygen supply node, running the central line through the center of the helix and having a second end attached to a control element;
controlling the central line with a control element;
operating the control element by a trigger allowing the central line to unwind while pulling the accordion support structure open; and
operating the control element by a trigger allowing the central line to rewind with a relatively light force while pushing the accordion support structure closed.

5. The method of claim 4, further comprising a chest-harness:
attaching a chest-harness safety-feature to the chest of a patient;
attaching the helical accordion support structure to the chest-harness; and
preventing severe stress on the oxygen supply line in the event the central control line element is dropped.

6. The method of claim 4 further comprising:
forming a kit for adapting a screw tightened bracket means to attach to an oxygen supply node; and
providing an anchor for the central line.
